(19) United States
(12) Patent Application Publication
(10) Pub. No.: US 2002/0019656 A1
(43) Pub. Date: Feb. 14, 2002

(54) Apparatus for water activation

(76) Inventors: Nataliya N. Lebedeva, Moscow (RU); Oleg V. Betsky, Frjazion (RU); Sergei V. Posmitny, Moscow (RU); Victor D. Kotov, Frjazion (RU)

Correspondence Address: GREENWALD & BASCH, LLP, SUITE 2490, 349 WEST COMMERCIAL STREET, EAST ROCHESTER, NY, 14445, US

(21) Appl. No.: 09/918,077

(22) Filed: Jul. 30, 2001

(30) Foreign Application Priority Data
Aug. 1, 2000 (RU) 2000120253

(51) Int. Cl. A61N 5/04

(52) U.S. Cl. 607/101; 607/156

(57) ABSTRACT

An apparatus for treating a body of water with a floating platform, a device for generating millimeter wave electromagnetic radiation connected to the floating platform, a transmitting antenna located below the floating platform, a receiving antenna located above the floating platform, a waveguide for conveying the millimeter wave electromagnetic energy to the transmitting antenna, and a power supply.

### CROSS-REFERENCE TO RELATED PATENT APPLICATIONS

Priority for this patent application is based upon applicants' Russian Federation patent application number 2000120253, filed on Aug. 1, 2000.

1. Field of the Invention

An apparatus for water treatment comprised of a floating platform disposed on the surface of the water, and a radiating antenna disposed below the surface of the water.

2. Background of the Invention

Millimeter waves have wavelengths of from about 10 to about 10 millimeters, corresponding to frequencies of from about 300 to about 30 gigahertz. In recent years, a substantial amount of research has been conducted regarding the biological and medical effects of such millimeter waves. See, e.g., an article by A. G. Pakhomov et al. entitled “Current state and implications of research on biological effects of millimeter waves: A review of the literature,” published in 1998 in Bioelectromagnetics, 19(7), at pages 393-413.

Today millimeter wave therapy, also known as “extremely high frequency therapy,” has become an approved and accepted method of medical treatment in Russia and many former Soviet republics. More than 2,000 physicians from all over Russia have completed formal educations courses in Moscow on the medical uses of millimeter waves; the method is currently used in more than 1,5000 hospitals and clinics in the Russian Federation; more than 1,000,000 patients have undergone this treatment; and more than 10,000 millimeter wave devices have been sold to research and clinical institutions. See, e.g., a paper by A. Yu. Lebedeva entitled “Millimeter waves in clinical practice in Russia: a Review” that was presented on Oct. 31, 2000 in Zvenigorod, Russia at the 12^{th} Russian Symposium on Millimeter Waves in Medicine and Biology.

It has been determined that low intensity millimeter waves (with power levels of less than about 11 milliwatts per square centimeter) have effects on cell growth and proliferation, activity of enzymes, the function of excitable membranes, periperal receptors, and other biological systems. See, e.g., the aforementioned 1998 article by A. G. Pakhomov et al.

It has also been determined that, in animals and humans, local millimeter wave exposure has stimulated tissue repair and regeneration, alleviated stress reactions, and facilitated recovery in a wide range of diseases. See, e.g., an 1999 article by N. N. Lebedeva and T. I. Kotorovskaya entitled “Experimental and clinical studies in the field of biological effects of millimeter waves” (review, part 1) published in Russian in Millimetrovye Volny v. Biologii I Meditsine (“Millimeter Waves in Medicine and Biology”), 3(15), pages 3-14.

It is an object of this invention to provide an apparatus for treating a body of water to enhance to life activity of various kinds of flora and fauna that inhabit the body of water.

### SUMMARY OF THE INVENTION

In accordance with this invention, there is provided an apparatus for treating a body of water which is comprised of a floating platform, means for generating millimeter wave electromagnetic radiation connected to said floating platform, a transmitting antenna disposed below said floating platform, a receiving antenna disposed above said floating platform, waveguide means for conveying said millimeter wave electromagnetic energy from said means for generating millimeter wave electromagnetic radiation to said transmitting antenna, and a power supply operatively connected to said means for generating said millimeter wave electromagnetic radiation.

### BRIEF DESCRIPTION OF THE DRAWINGS

The invention will be described by reference to the following drawings, in which like numerals refer to like elements, and in which:

FIG. 1 is a schematic representation of one preferred device of this invention.

### DESCRIPTION OF THE PREFERRED EMBODIMENT

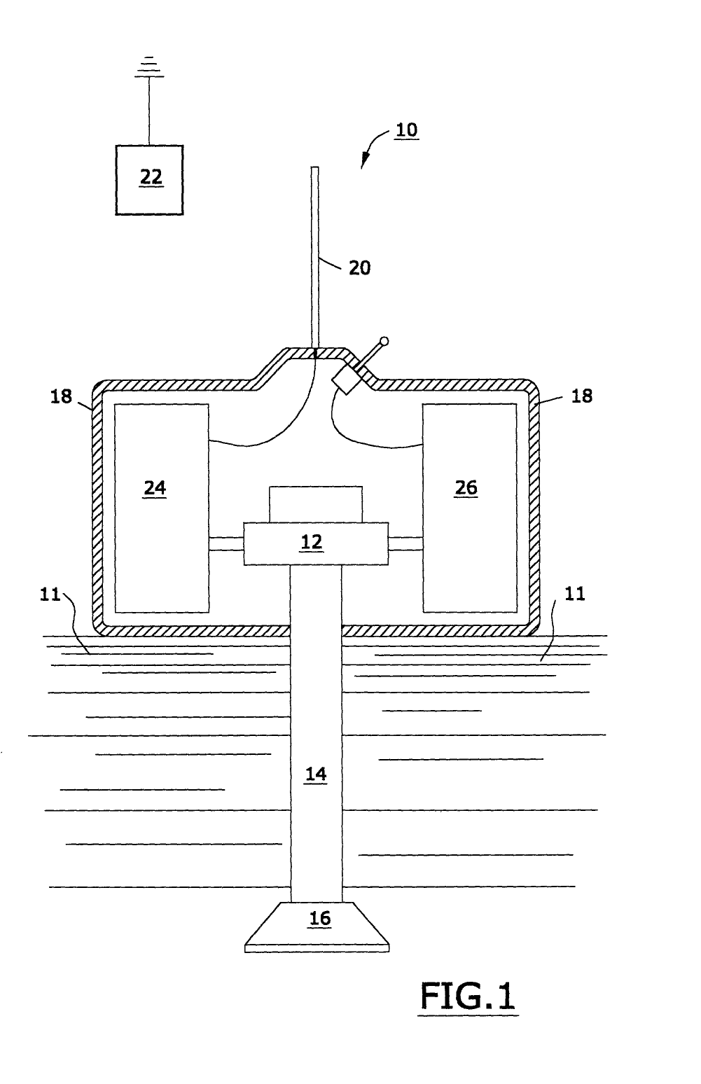

In the process and apparatus of this invention, a millimeter wave generator is used. These generators are well known to those skilled in the art and are commercially available. Thus, e.g., referring to U.S. Pat. No. 3,596,695, the entire disclosure of which is hereby incorporated by reference into this specification, it is disclosed that “Referring now to FIG. 1, there is illustrated in block form an apparatus embodying the present invention. The apparatus of FIG. 1 includes a variable microwave generator 10. The microwave generator 10 is continuously variable over a predetermined frequency range as indicated by the arrow 11. Such microwave generators are readily obtainable in the trade. For example, Model No. 440XXH represents a series of microwave generators obtainable from Hughes Aircraft Company. By way of example, Model No. 44076H is a millimeter wave generator having a 3 mW output over a 10 GHz bandwidth between 60 to 90 GHz and includes an isolator. Other models are available with other frequency ranges and with similar power outputs.”

By way of further illustration, U.S. Pat. No. 6,101,015 discloses a microwave or millimeter wave generator. U.S. Pat. No. 5,777,572 discloses a gyrotron oscillator millimeter wave generator for producing high power millimeter wave beams for jamming and/or damaging electronic equipment; the generator of this patent produces 20 millisecond megawatt pulses at a frequency of from 100 to 140 gigahertz. U.S. Pat. No. 5,760,397 discloses a millimeter wave imaging system. U.S. Pat. No. 5,507,791 discloses a millimeter wave generator producing radiation with a frequency of from 40 to 70 gigahertz. U.S. Pat. No. 5,379,309 discloses a photonic down conversion system which employs a millimeter wave generator. In FIG. 3 (element 15) of U.S. Pat. No. 5,344,099, a millimeter wave generator is shown. U.S. Pat. No. 5,227,800 discloses a millimeter wave generator used to illuminate objects in the field of view of a millimeter wave camera. A millimeter wave generator is mentioned in claim 16 of U.S. Pat. No. 5,223,352. U.S. Pat. No. 5,152,286 discloses a spark (noise) generator for producing extremely high frequency (EHF) electromagnetic radiation. U.S. Pat. No. 5,131,409 discloses a microwave resonance therapy generator. U.S. Pat. No. 4,306,174 discloses a radio wave generator for ultra-high frequencies. U.S. Pat. No. 4,286,230 discloses a near millimeter wave generator with a dielectric cavity. The entire disclosure of each of these United States patents is hereby incorporated by reference into this specification.

FIG. 1 is a schematic diagram of one preferred water treatment apparatus 10 of the invention which, in the embodiment depicted, is shown floating on water 11; the drawing of FIG. 1 is not necessarily drawn to scale.

Apparatus 10 is comprised of a generator 12 which is adapted to generate millimeter wave energy at a frequency of from about 35 to about 75 gigahertz and, preferably, at a frequency of from about 46 to about 65 gigahertz. In a preferred embodiment, the wavelengths of the energy produced by generator 12 are from about 4.6 to about 6.7 millimeters.

The energy produced by generator 12 is channeled through waveguide 14 and transmitted through antenna 16. In the embodiment depicted, waveguide 14 is securely attached to the platform 18, which is floating on water 11.

One may use any waveguide known to efficiently couple gigahertz frequency energy from a generator to an antenna. The waveguide 14 is preferably a hermetic waveguide, i.e., it prevents the water 11 from entering it an contacting the radiation emitted by generator 12.

One may use any of the hermetic waveguide designs known to those skilled in the art. Thus, by way of illustration and not limitation, one may use the hermetic waveguide disclosed in U.S. Pat. No. 5,202,648, the entire disclosure of which is hereby incorporated by reference into this specification. This patent discloses and claims a waveguide-to-microstrip transition module for transmitting captured electromagnetic energy between a waveguide and signal processing circuitry, the module comprising: a base including at least one waveguide through which said electromagnetic energy is transmitted into the module to the signal processing circuitry, said at least one waveguide locatable anywhere on said base; a circuit board having one continuous side hermetically sealed to the base and the opposite side including at least one microstrip connected to signal processing circuitry and cooperatively oriented with said at least one waveguide; at least one backshort, mounted on said opposite side of the circuit board, said backshort cooperatively associated with said at least one waveguide and said at least one microstrip; a housing containing the base and circuit board; and a cover hermetically sealed to the housing to enclose the circuit board in the housing.

Referring again to FIG. 1, and in the preferred embodiment depicted, the waveguide 14 preferably has a cylindrical housing. In one aspect of this embodiment, a quasi-optical fiber is disposed within a cylindrical metal wavegide.

The waveguide 14 is connected to a platform 18. In the preferred embodiment depicted in FIG. 1, the platform 18 is in the shape of a truncated cylinder which, preferably, has a diameter of about 5 centimeters and a height of about 5 centimeters.

The platform 18 is adapted to float upon water 11; thus, it preferably has a specific gravity of less than about 1 gram per cubic centimeter and, preferably, from about 0.1 to about 0.8 grams per cubic centimeter.

One may use any material with a suitable specific gravity which will float upon water 11. Thus, by way of illustration and not limitation, one may use foam materials. See, e.g., pages 349-351 of George S. Brady et al.'s “Materials Handbook,” Thirteenth Edition (McGraw-Hill, Inc., New York, N.Y., 1991)

In one preferred embodiment, the foam used is “STYROFOAM,” an expanded cellular polystyrene expanded into a muticellular mass 42 its original size and sold by the Dow Chemical Corporation of Midland, Mich.

Referring again to FIG. 1, a receiving antenna 20 is adapted to receive signals from a remote transmitter 22, which communicates with device 10 by means of antenna 20. Signals picked up by antenna 20 are conveyed via line 22 to controller 24. Controller 24 preferably comprises means for regulation of voltage, amperage, power, frequency, mode, phase, etc. Thus, e.g., the controller 24 can turn the power off and on, can vary the amount of power supplied to to the generator 12, can vary the type of power supplied to generator 12, etc.

The apparatus 10 also comprises a power supply 26 which, in the embodiment, depicted, preferably comprises a multiplicity of batteries. In one aspect of this embodiment, four batteries each delivering 1.2 volts are connected in series to provide 4.8 volts of direct current.

As will be apparent to those skilled in the art, the use of autonomous power source 26 with control unit 24, located on platform 18, makes it possible to control the apparatus 10 floating on the surface of, e.g., a water reservoir from the shore. Thus, e.g., the transmitter 22 and/or a transceiver 22 can switch unit 10 off and on, can change its mode of operation, etc.

In one preferred embodiment, the generator 12 works with a constant power supply of 5 volts and a current of less than about 100 milliamperes and produces continous waves in the millimeter wave range of from about 4.6 to about 6.7 millimeters. In one aspect of this embodiment, the radio frequency energy so produced is produced by a Gunn diode.

The power of the energy transmitted through antenna 16 is preferably from about 20 to about 60 milliwatts and, more preferably, from about 30 to about 50 milliwatts.

In one embodiment, the antenna 16 emits continuous, non harmonic oscillations. In one aspect of this embodiment, the output antenna is a microwave horn antenna. Reference may be had, e.g., to U.S. Pat. Nos. 5,779,844, 5,705,022, 5,657,033, 5,565,067, 5,497,100, 5,428,360, 5,418,463, 5,384,543, 5,302,208, 5,200,757, 5,012,250, 4,957,061, 4,878,061, 4,563,687, 4,373,161, 4,027,303, 3,968,495, and the like. The entire disclosure of each of these United States patents is hereby incorporated by reference into this specification.

Referring again to FIG. 1, and in the preferred embodiment depicted therein, the antenna 16 preferably is covered by a plastic material to render it waterproof. In one aspect of this embodiment, the antenna 16 preferably emits a toroidal polarization pattern.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the following claims.

We claim:

1. An apparatus for treating a body of water which is comprised of a floating platform, means for generating millimeter wave electromagnetic radiation connected to said floating platform, a transmitting antenna disposed below said floating platform, a receiving antenna disposed above said floating platform, waveguide means for conveying said millimeter wave electromagnetic energy from said means for generating millimeter wave electromagnetic radiation to said transmitting antenna, and a power supply operatively connected to said means for generating said millimeter wave electromagnetic radiation.

2. The apparatus as recited in claim 1, wherein said floating platform is comprised of material with a specific gravity of less than about 1.0 gram per cubic centimeter.

3. The apparatus as recited in claim 2, wherein said floating platform consists essentially of material with a specific gravity of less than about 1.0 gram per cubic centimeter.

4. The apparatus as recited in claim 3, wherein said floating platform consists essentially of foam material.

5. The apparatus as recited in claim 4, wherein said foam material is expanded cellular polystyrene.

6. The apparatus as recited in claim 1, wherein said transmitting antenna is a microwave horn antenna.

7. The apparatus as recited in claim 6, wherein said transmitting antenna emits continuous waves in the millimeter wave range of from about 4.6 to about 6.7 millimeters.

8. The apparatus as recited in claim 7, wherein said transmitting antenna emits continuous waves with a power of from about 20 to about 60 milliwatts.

9. The apparatus as recited in claim 8, wherein said transmitting antenna emits continuous waves with a power of from about 30 to about 50 milliwatts.

10. The apparatus as recited in claim 9, wherein said transmitting antenna is covered with a waterproof, plastic material.

11. The apparatus as recited in claim 1, wherein said power supply is comprised of a multiplicity of batteries.

12. The apparatus as recited in claim 11, wherein said power supply supplies about 5.0 volts of direct current.

13. The apparatus as recited in claim 1, wherein said waveguide is a hermetic waveguide.

14. The apparatus as recited in claim 13, wherein said waveguide is comprised of a cylindrical housing.

15. The apparatus as recited in claim 14, wherein an optical fiber is disposed within said cylindrical housing.

16. The apparatus as recited in claim 1, wherein said floating platform is in the shape of a truncated cylinder.
